(19) United States
(12) Patent Application Publication
(10) Pub. No.: US 2023/0296597 A1
(43) Pub. Date: Sep. 21, 2023

(54) Devices and Methods for Detection of Viruses from Exhaled Breath

(71) Applicant: Northeastern University, Boston, MA (US)

(72) Inventors: Nian-Xiang Sun, Winchester, MA (US); Shadi Emam, Boston, MA (US); Mehdi Nasrollahpour, Boston, MA (US)

(21) Appl. No.: 18/040,920

(22) PCT Filed: Jan. 20, 2021
(86) PCT No.: PCT/US2021/014248

§ 371 Date: Feb. 7, 2023

Related U.S. Application Data
(60) Provisional application No. 63/065,462, filed on Aug. 13, 2020.

(51) Int. Cl. G01N 33/543; G01N 33/569; G01N 27/12

(52) U.S. Cl. CPC G01N 33/5438; G01N 33/56983; G01N 27/125; G01N 2333/165; G01N 2333/08

(57) ABSTRACT

Devices and methods for the detection of an analyte, e.g, from exhaled breath or air are provided. A sensor includes a polymer layer molecularly imprinted for the analyte, a metal layer, and an electrocatalytic layer disposed between the polymer layer and the metal layer. The electrocatalytic layer is functionalized with at least one chemical compound that provides for noncovalent interaction with the analyte. The sensor further includes electrodes in operative arrangement with the polymer layer and configured to provide a signal indicative of a resistance. A change in resistance of the device can indicate the presence of the analyte in the sample.

### RELATED APPLICATIONS

This application claims the benefit of U.S. Provisional Application No. 63/065,462, filed on Aug. 13, 2020. The entire teachings of the above application are incorporated herein by reference.

### GOVERNMENT SUPPORT

This invention was made with government support under Grant Number ECCS 2031142 awarded by the National Science Foundation. The government has certain rights in the invention.

### BACKGROUND

In the last twenty years, several viral epidemics, such as the severe acute respiratory syndrome coronavirus (SARS-CoV) in 2002 to 2003, the H1N1 influenza in 2009, and the Middle East respiratory syndrome coronavirus (MERS-CoV) in Saudi Arabia in 2012, have been identified. Severe acute respiratory syndrome coronavirus 2 (SAR S-CoV-2) is a new virus that causes COVID-19, which has killed hundreds of thousands of people so far. The respiratory symptoms of COVID-19 typically appear an average of 5-6 days after exposure but may appear in as few as 2 days or as many as 14 days after exposure, according to the U.S. Centers for Disease Control and Prevention (CDC). During all these times, the disease is epidemic.

Health officials first believed that the virus SA RS-CoV-2 is transported only through droplets that are coughed or sneezed out, either directly or onto objects. But some scientists say there is preliminary evidence that airborne transmission in which the disease spreads in the much smaller particles from exhaled air, known as aerosols, is occurring. Compared with droplets, which are heftier and thought to travel only short distances after someone coughs or sneezes before falling to the floor or onto other surfaces, aerosols can linger in the air for longer and travel further. Aerosols are more likely to be produced by talking and breathing.

Routine confirmation of cases of COVID-19 is based on detection of unique sequences of virus RNA by nucleic acid amplification tests (NAAT), such as real-time reverse-transcription polymerase chain reaction (rRT-PCR), with confirmation by nucleic acid sequencing when necessary.

There exists a need for integrated, random-access, point-of-care devices that can provide for accurate diagnosis of SARS-CoV-2 infections.

### SUMMARY

Methods and devices for detecting anaytes. e.g., viruses, such as SARS-CoV-2, from samples, such as droplets or aerosols from exhaled breath, are provided. The methods and devices described can provide for rapid, non-invasive, and specific detection of a viral infection.

A sensor includes a polymer layer molecularly imprinted for an analyte, a metal layer, an electrocatalytic layer disposed between the polymer layer and the metal layer, and electrodes in operative arrangement with the polymer layer and configured to provide a signal indicative of a resistance. The electrocatalytic layer is functionalized with at least one chemical compound that provides for noncovalent interaction with the analyte.

The analyte can be a receptor-binding domain (RBD) of a virus, such as SARS1, SARS2, SARS-CoV-2, and Ebola, including any mutations thereof (e.g., the SARS-CoV-2 virus can be the SARS-D614D strain, SARS-D614G strain, etc.). For example, the analyte can be a glycoprotein, such as a glycosylated spike protein. The glycosylated spike protein can be a spike protein that binds to an angiotensin-converting enzyme 2 (ACE2) receptor.

The analyte can be, for example, a protein, and the at least one chemical compound of the electrocatalytic layer can mimic a feature of a cell receptor for the protein (e.g., an angiotensin-converting enzyme 2 (ACE2) receptor). For example, the at least one chemical compound can comprise 1-pyrenebutyric acid n-hydroxysuccinimide ester (PBSE), cysteamine, or a combination thereof.

The metal layer of the detector can comprise any of chromium, platinum, gold, nickel, cobalt, tungsten, rhodium, iridium, silver or tin, or alloys thereof. The metal layer can be disposed on a silicon substrate. A detector can include a sensor and a voltage source configured to apply a voltage to the polymer layer. The detector can further include an ohmmeter in operative arrangement with the electrodes and configured to output a measurement of the resistance

A method of detecting an analyte includes exposing a sensor or a detector to a sample and measuring the resistance of the device as the polymer layer is exposed to the same. The measured resistance can be indicative of a presence of the analyte in the sample. The sample can comprise, for example, an aerosol or droplets from exhaled breath.

A method of detecting an infection, e.g., a viral infection, in a subject includes exposing the sensor or a detector to a sample obtained from the subject and measuring the resistance of the device as the polymer layer is exposed to the biological sample. The analyte can be a receptor-binding domain of a virus and the measured resistance is indicative of the viral infection in the subject.

A method of detecting an analyte includes measuring a resistance of a molecularly imprinted polymer exposed to a sample. The molecularly imprinted polymer is imprinted for the analyte and in operative arrangement with an electrocatalyst and a metal. The electrocatalyst is functionalized with at least one chemical compound that provides for noncovalent interaction with the analyte and is disposed between the molecularly imprinted polymer and the metal. The measured resistance is indicative of a presence of the analyte in the sample.

The method can further include comparing the measured resistance to an inherent resistance of the molecularly imprinted polymer in arrangement with the electrocatalyst and the metal for determination of whether the measure resistance is indicative of the presence of the analyte. For example, if the measured resistance is greater than the inherent resistance, the measured resistance indicates that the analyte is present in the sample. The sample can be in an aerosol or droplet form in exhaled breath.

A method of manufacturing a sensor includes depositing an electrocatalytic layer onto a metal layer, functionalizing the electrocatalytic layer with at least one chemical compound that provides for noncovalent interaction with an analyte of interest, and forming a molecularly imprinted polymer on the electrocatalytic layer, the molecularly imprinted polymer imprinted for the analyte.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a depiction of an example sensor for detection of a virus.

FIG. 2 is a schematic illustrating formation and use of a functionalized sensor.

FIGS. 3A-3D are graphs displaying the results of four non-functionalized sensors to low (10 ng/μl) and high (100 ng/μl) concentrations of four different viruses, including Ebola (FIG. 3A), SARS1 (FIG. 3B), SARS-Cov2-D614D (FIG. 3C), and SARS-Cov2-D614G (FIG. 3D).

FIGS. 4A-4B are graphs displaying the results of two double-functionalized sensors to low (10 ng/μl) and high (100 ng/μl) concentrations of SARS2-D614D (FIG. 4A) and SARS2-D614G (FIG. 4B).

FIGS. 5A-5B are graphs displaying the results of a non-functionalized sensor (FIG. 5A) and a cysteamine-functionalized sensor (FIG. 5B) to SARS2.

FIG. 6 is a graph displaying the results of a cysteamine-functionalized sensor to SARS2.

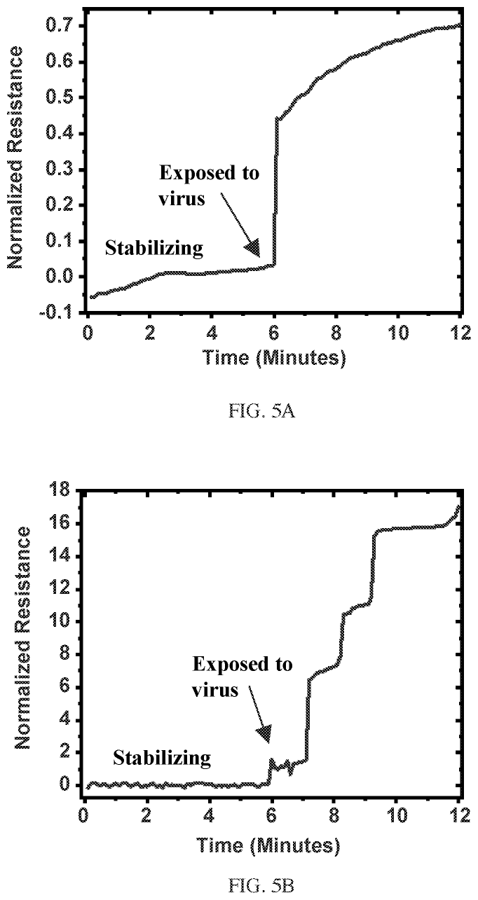

FIG. 7 is a schematic of an example detector.

FIG. 8 is a graph displaying the results of a double-functionalized sensor to samples of 1 μg/ml and 300 μg/ml concentrations of spike proteins of SARS-Cov2-D614D.

### DETAILED DESCRIPTION

A description of example embodiments follows.

As used herein, the singular forms “a,” “an” and “the” include plural referents unless the context clearly dictates otherwise. Thus, for example, reference to “a layer” can include a plurality of layers. Further, the plurality can comprise more than one of the same layer, or a plurality of different layers.

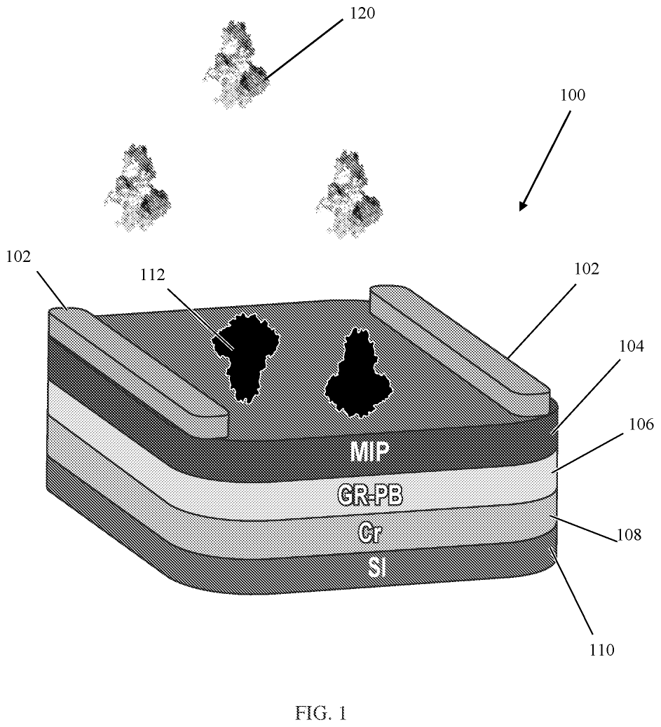

An example of a sensor for the detection of a virus is shown in FIG. 1. The sensor 100 includes a polymer layer 104 that is molecularly imprinted for an analyte 120, an electrocatalytic layer 106, and a metal layer 108. The polymer layer 104 comprises imprints 112 selective for the analyte 120. Electrodes 102 are in operative arrangement with the polymer layer 104 for measuring a resistance through the device. Optionally, the sensor includes a substrate layer 110.

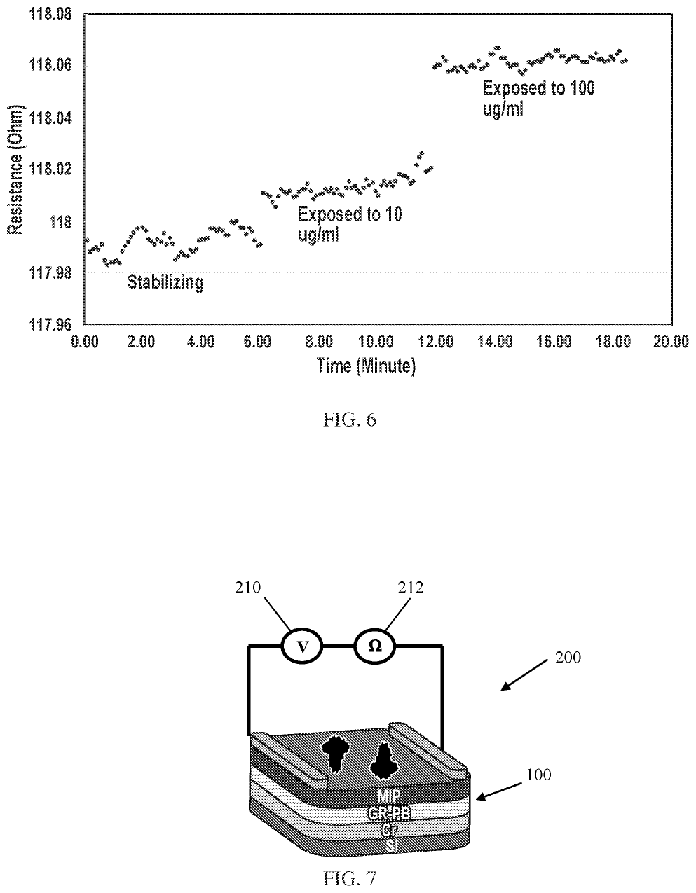

As shown in FIG. 7, a detector 200 can include a sensor 100, a voltage source 210 configured to apply a voltage to the polymer layer, and an ohmmeter 212 in operative arrangement with the electrodes and configured to output a measurement of the resistance through the device.

Examples of sensors comprising molecularly imprinted polymers are further described in U.S. Pub. No. 2019/0313944, titled “Molecularly-Imprinted Electrochemical Sensors,” the entire teachings of which are incorporated herein by reference. As described therein, there has been success in imprinting such devices for small molecules.

Molecular imprinting for detection of a protein, such as a spike protein associated with a virus, poses several challenges due to the relatively large size, chemical and structural complexity, and environmental instability of proteins. Due to their relatively large size, proteins are unable to diffuse into, or out of, many of the imprinted cavities within a bulk molecularly-imprinted polymer (MIP). Furthermore, proteins generally require specific environmental conditions (e.g., temperature around 37° C. and no surfactant) in order to remain in their native form.

To provide for improved detection of analytes comprising larger molecules, such as proteins, the electrocatalytic layer of a sensor can be functionalized with at least one chemical compound that provides for noncovalent interaction with the analyte. Functionalization of the electrocatalytic layer can provide, for example, for an increased number of noncovalent interactions and/or an increased strength of noncovalent interaction between the analyte (including its template molecule) and the polymer.

As used herein, the term “functionalized” refers to having a molecule or a particle attached to a surface so as to provide for a modified physical or chemical property.

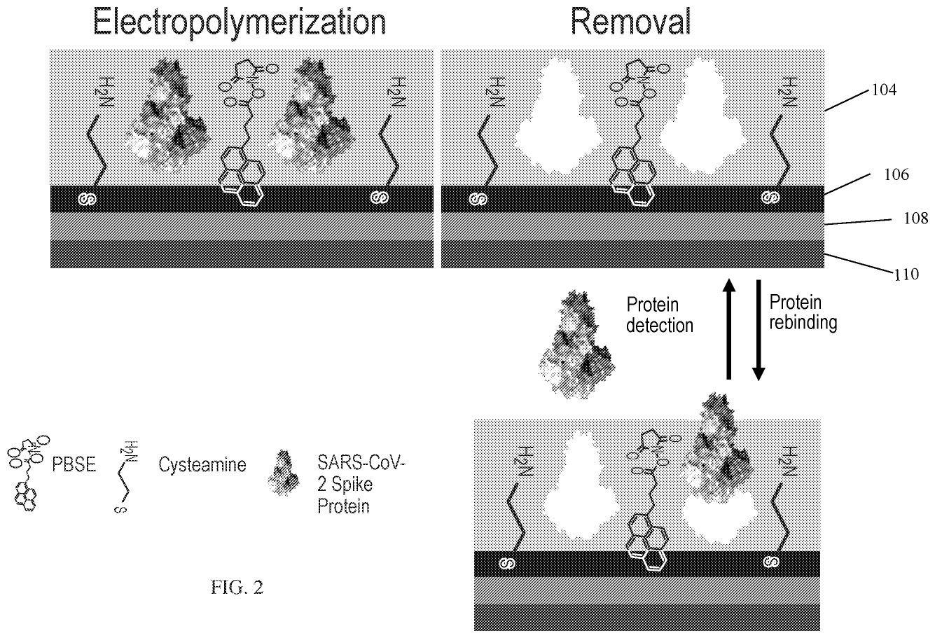

An example of a sensor comprising a functionalized electrocatalytic layer is shown in FIG. 2. As illustrated, the electrocatalytic layer comprises graphene and potassium ferrocyanide (Gr-PB). Potassium ferrocyanide is alternatively referred to as Prussian Blue. The Gr-PB layer is functionalized with two chemical compounds, in particular, cysteamine and 1-pyrenebutyric acid n-hydroxysuccinimide ester (PBSE), to assist in the detection of the SARS-CoV-2 spike protein. The cysteamine and PBSE molecules extend from the Gr-PB layer into the polymer layer, which is imprinted for a protein, which, in this example, is the SARS-CoV-2 spike protein.

The SARS-CoV-2 spike (S protein) glycoprotein is a key target for vaccines, therapeutic antibodies, and diagnostics. The virus uses the glycosylated spike (S) protein to gain entry to host cells. The pre-fusion process is triggered when the S1 subunit of the protein binds to a host cell receptor. After receptor binding destabilizes, shedding of the S1 subunit occurs and transition of the S2 subunit to a stable post-fusion conformation results.

Angiotensin-converting enzyme 2 (ACE2) is the cellular receptor for SARS-CoV and the new coronavirus, SARS-CoV-2. The SARS-CoV-2 receptor binding domain (RBD) exhibited significantly higher binding affinity to the ACE2 receptor than the SARS-CoV RBD. Tai, W., He, L., Zhang, X. et al. Characterization of the receptor-binding domain (RBD) of 2019 novel coronavirus: implication for development of RBD protein as a viral attachment inhibitor and vaccine. Cell Mol Immunol 17, 613-620 (2020). The recombinant RBD protein was found to block binding and, hence, attachment of SARS-CoV-2 RBD and SARS-CoV RBD to ACE2-expressing cells, thus inhibiting their infection of cells. Id. Researchers have determined the crystal structure of the SARS-CoV-2 RBD in complex with human ACE2 (hACE2) and found that several residue changes in SARS-CoV-2 RBD stabilize two virus-binding hotspots at the RBD/hACE2 interface, which enhances the binding between SARS-CoV-2 RBD and hACE2. Shang, J., Ye, G., Shi, K. et al. Structural basis of receptor recognition by SARS-CoV-2. Nature 581, 221-224 (2020); and Prabakaran P, Xiao X, Dimitrov D S. A model of the ACE2 structure and function as a SARS-CoV receptor. Biochem Biophys Res Commun. 2004 Jan. 30; 314(1):235-41.

In another study, the COVID-19 spike binding site of the cell-surface receptor (Glucose Regulated Protein 78 (GRP78)) was predicted using combined molecular modeling docking and structural bioinformatics. Also, the COVID-19 spike protein has been modeled using its counterpart, the SARS spike. Ibrahim I M, Abdelmalek D H, Elshahat M E, Elfiky A A. COVID-19 spike-host cell receptor GRP78 binding site prediction. J Infect. 2020 May; 80(5):554-562. Further, the hydrogen bonds and salt bridges at the 2019-nCoV RBD/ACE2 and SARS-CoV RBD/ACE2 interfaces have been identified. Lan, J., Ge, J., Yu, J. et al. Structure of the SARS-CoV-2 spike receptor-binding domain bound to the ACE2 receptor. Nature 581, 215-220 (2020). There are 17 hydrogen bonds and 1 salt bridge at the 2019-nCoV RBD/ACE2 interface, and 12 hydrogen bonds and 2 salt bridges at the SARS-CoV RBD/ACE2 interface. Another shared feature of the 2019-nCoV RBD/ACE2 interface and the SARS-CoV RBD/ACE2 interface is the involvement of multiple tyrosine residues in forming hydrogen-bonding interactions with the polar hydroxyl group.

Molecular imprinting is a polymer chemistry approach that can be used for design and production synthetic receptors. Molecularly imprinted polymers (MIPs) can serve as crude mimics of native receptors. Molecular imprinting has also been used in the production of artificial antibodies. For the production of artificial antibodies, monomers with a functional group capable of forming favorable interactions with a molecule of interest, called the template, are used as building blocks instead of amino acids. In the production of antibodies, cells incorporate amino acids in an exact order based on genetic code, while in the preparation of MIPs, self-assembly of the functional monomers with the template is relied upon.

As noted above, sensors comprising molecularly imprinted polymers have been successfully made and used for the detection of small molecules, including volatile organic compounds. Examples of sensors comprising molecularly imprinted polymers and methods of making such sensors are further described in U.S. Pub. No. 2019/0313944, titled “Molecularly-Imprinted Electrochemical Sensors,” the entire teachings of which are incorporated herein by reference. In short, molecular imprinting is a technique in which a thin layer of polymer is electrochemically formed around template molecules of interest. The template molecules are then removed after polymerization. As such, an imprint having the size and shape of the template molecule is left in the polymer matrix. This results in a lock (sensor cavity) and key (template molecule) relationship of high specificity.

To provide for the detection of viruses, such as human coronavirus (HCoV-NL63) and human severe acute respiratory syndrome coronaviruses (SARS-CoV and SARS-CoV-2), molecularly imprinted electrochemical sensors can be functionalized to better mimic native receptors that engage with viral proteins, e.g., fusion proteins, and provide for improved detection performance. By mimicking chemical bonding between the template molecule and the sensor during fabrication (and consequently, between an analyte and the sensor during use), enhanced sensitivity and specificity can be obtained.

For example, each of the SARS1, SARS2, SARS-CoV-2, and Ebola viruses contains at its surface glycoproteins that bind to receptors on a cell surface. As described above, ACE2 is the cellular receptor for SARS-CoV and the new coronavirus, SARS-CoV-2. Cysteamine is a decarboxylated derivative of cysteine, which can provide for increased binding affinity for glycoproteins due to its amine group, and 1-Pyrenebutyric acid N-hydroxysuccinimide ester (PBSE) is structurally very similar to an ACE2 receptor binding domain (RBD) that binds with the virus. To resemble and utilize the affinity of ACE2 with SARS-CoV-2, cysteamine and PBSE, each alone or in combination, can be included in a sensor through functionalization of the electrocatalytic layer, as shown in the example sensor of FIG. 2.

ACE 2 is a functional receptor for the spike glycoprotein of the human coronavirus HCoV-NL63 and the human severe acute respiratory syndrome coronaviruses, SARS-CoV and SARS-CoV-2. The spike (S) protein of SARS-CoV-2 is an important component in the receptor recognition and cell membrane fusion process. The S protein includes two subunits, S1 and S2. The S1 subunit contains a receptor-binding domain that recognizes and binds to the host receptor ACE2. The S2 subunit mediates viral cell membrane fusion. This glycoprotein has the size of 180-200 kDa and is covered with polysaccharide. Huang, Y., Yang, C., Xu, Xf. et al. Structural and functional properties of SARS-CoV-2 spike protein: potential antivirus drug development for COVID-19. Acta Pharmacol Sin 41, 1141-1149 (2020).

Cysteamine and PBSE have each been shown as being capable of attaching to graphene. Cysteamine-functionalized graphene has been applied to detect Hg(2+) and carbohydrate antigen 125 (CA-125). See Zhou, Heng et al. Sensitive and selective voltammetric measurement of Hg2+ by rational covalent functionalization of graphene oxide with cysteamine. The Analyst. 137. 305-8 (2012); and Rebelo, Tânia et al. Molecularly imprinted polymer SPE sensor for analysis of CA-125 on serum. Analytica Chimica Acta. 1082 (2019).

PBSE-functionalized graphene has been applied to detect SARS-CoV-2, with SARS-CoV-2 spike antibody conjugated onto a graphene sheet via PBSE. The PBSE served as an interfacing molecule (i.e., a probe linker) to tether the antibody probe to the graphene sheet. See Seo, Giwan et al. Rapid Detection of COVID-19 Causative Virus (SARS-CoV-2) in Human Nasopharyngeal Swab Specimens Using Field-Effect Transistor-Based Biosensor. ACS Nano 2020 14 (4), 5135-5142.

Fabrication and use of an example of a functionalized sensor is shown in FIG. 2. In particular, the Gr-PB layer is functionalized with both cysteamine and PBSE, and electropolymerization of the polymer layer occurs in the presence of the template molecule (e.g., SARS-CoV-2 Spike Protein). The template molecule is subsequently removed. Upon exposure to a sample containing the SARS-CoV-2 virus, the spike protein of the virus rebinds to the sensor. A resistance through the sensor can be measured, with a change in resistance, or a comparison of a measured resistance to a threshold value, indicating the presence of the virus in the sample.

As further described in the Examples herein, such sensors can provide significantly improved sensitivity over non-functionalized sensors. The sensors can be used to rapidly detect the presence of viruses, such as SARS-CoV-2, in approximately 1-2 seconds with ˜100% sensitivity and 100% specificity based on demonstrated results. The electrochemical sensors, with molecularly imprinted polymer and functionalization, show part per quintillion (10\hat{ }−18) sensitivity, fast sensor response, recovery in approximately 1-2 seconds, and ultra-high specificity. The sensors can be included in breathalyzer devices for detection of the virus in a subject. Alternatively, the sensors can be used for air contamination monitoring. For example, sensors as described herein can be disposed within an air-circulation device to monitor the presence of aerosolized virus in a room.

The sensor can be molecularly imprinted for an analyte that is, e.g., a receptor-binding domain of a virus. For example, the analyte can be a protein, such as a glycoprotein or other viral fusion protein, located on a virus. The glycoprotein can be a glycosylated spike protein, such as one that binds to an angiotensin-converting enzyme 2 (ACE2) receptor. The virus can be a human coronavirus, including any mutations thereof (e.g., the SARS-CoV-2 virus can be SARS-D614D, SARS-D614G, etc.). The polymer layer of the sensor can be imprinted for one or more analytes. For example, the polymer layer can be selective for spike proteins of both the SARS-D614D and SARS-D614G mutations. For structurally-similar analytes (e.g., the spike proteins of the SARS-D614D and SARS-D614G mutations), molecular imprinting for one of the analytes may allow for the sensor to provide for the detection of one or more other, e.g., related, analytes. Alternatively, the polymer layer can be imprinted with template molecules of more than one structurally-distinct analyte.

As used herein, “molecularly imprinted polymer,” or “MIP,” refers to a polymer created using molecular imprinting technology. The polymeric matrix of a “molecularly imprinted polymer” is characterized by molecular recognition sites (i.e., “imprints”) specific to one or more analytes that enable the MIP to selectively bind the one or more analytes. A polymer that is imprinted “for” an analyte is a polymer that includes molecular recognition sites selective to that analyte. The molecularly imprinted polymer can comprise a conductive polymer, such as polypyrrole.

It will be understood that molecularly imprinted polymers, like polymers generally, are composed substantially or entirely of repeated monomeric subunits. Polymers can, therefore, be said to be derived from the monomers that make up the repeated monomeric subunits. For example, polypyrrole can be described as being derived from pyrrole. Examples of monomers from which a molecularly imprinted polymer can be derived include acrylic acid, methacrylic acid, 2-(trifluoromethly)acrylic acid, itaconic acid, p-vinylbenzoic acid, 2-acrylamido-2-methyl -1propansulfonic acid, divinylbenzene, 4-vinylbenzene boronic acid, 2-vinylpyridine, 4-vinylpyridine, N,N-diethylaminoethylmethacrylate, 1-vinylimidazole, allylamine, 4-(5-vinylimidazole), N-(2-aminoethyl)methacrylamide, N,N′-diethyl-4-styrylamidine, N,N,N -trimethylaminoethylmethacrylate, N-vinylpyrrolidone, urocanic ethyl ester, pyrrole, methyl methacrylate, 2-hydroxyethylmethacrylate, 4-ethyl styrene, acrylamide, methacrylamide, trans-3-(3-pyridyl)-acrylic acid, acrylonitrile and styrene, and any combination of the foregoing. In some aspects, the molecularly imprinted polymer is derived from acrylic acid, methacrylic acid, 2-(trifluoromethly)acrylic acid, itaconic acid, p-vinylbenzoic acid, 2-acrylamido-2-methyl -1propansulfonic acid, divinylbenzene, 4-vinylbenzene boronic acid, 2-vinylpyridine, 4-vinylpyridine, N,N-diethylaminoethylmethacrylate, 1-vinylimidazole, allylamine, 4-(5-vinylimidazole), N-(2-aminoethyl)methacrylamide, N,N′-diethyl-4-styrylamidine, N,N,N -trimethylaminoethylmethacrylate, N-vinylpyrrolidone, urocanic ethyl ester, pyrrole, methyl methacrylate, 2-hydroxyethylmethacrylate, 4-ethyl styrene, acrylamide, methacrylamide, trans-3-(3-pyridyl)-acrylic acid, acrylonitrile or styrene, or a combination of any of the foregoing. In a particular aspect, the molecularly imprinted polymer is derived from pyrrole.

The sensor can include, beneath or adjacent to the MIP layer, an electrocatalytic layer. The electrocatalytic layer can comprise one or more layers of graphene, either alone or in combination with a dopant, such as potassium ferrocyanide, which can assist in electron transfer between the MIP and graphene layers of a sensor. For example, a sensor can include 1 to about 30 layers of graphene, about 5 to about 20 layers of graphene, or about 5 to about 15 layers of graphene. The one or more layers of graphene can be in electrical communication with both the MIP layer and the metal layer.

As used herein, the term “electrocatalytic layer” refers to a layer comprising a material that can participate in an electrochemical reaction. The electrocatalytic layer can assist in transfer of electrons between the MIP layer and the metal layer. For example, when an analyte is trapped in the MIP layer, an extra electron can be transferred to the graphene layer, which causes a change in resistance of the device. Examples of suitable materials for the electrocatalytic layer include graphene and carbon nanotubes (CNT).

As used herein, “graphene” refers to a single-layer sheet of sp²-bonded carbon atoms arranged in a hexagonal, honeycomb lattice. Graphene typically contains defects, such as oxygen- or nitrogen-centered defects. “Graphene” is meant to encompass these species as well. Thus, as used herein, “graphene” includes graphene oxide (GO) and reduced graphene oxide (rGO, GR).

“Graphene oxide” or “GO” is graphene modified with oxygen-containing functional groups such as epoxides, carbonyls, carboxyls and alcohols. Typically, the carbon to oxygen ratio of graphene oxide is about three to about one. In some aspects, graphene is graphene oxide.

“Reduced graphene oxide,” “rGO” or “GR” is the product resulting from the reduction of graphene oxide. Reduced graphene oxide can be produced by a number of techniques known in the art, including the so-called Hummer method, wherein a solution of graphene oxide is exposed to hydrazine hydrate, and the solution is maintained at about 100° C. for about 24 hours. In some aspects, graphene is reduced graphene oxide.

In some aspects, graphene (e.g., reduced graphene oxide) comprises potassium ferrocyanide (also known as Prussian blue). Methods of making graphene comprising potassium ferrocyanide are known in the art and are further described in U.S. Pub. No. 2019/0313944, the entire teachings of which are incorporated herein by reference.

The electrocatalytic layer can be functionalized with at least one chemical compound that mimics a feature of a cell receptor for an analyte protein (e.g., an angiotensin-converting enzyme 2 (ACE2) receptor). For example, the electrocatalytic layer can be functionalized with 1-pyrenebutyric acid n-hydroxysuccinimide ester (PBSE), cysteamine, or a combination thereof. The chemical compound is thereby able to interact with the template molecule during the MIP-forming process and with analyte molecules during use of the sensor for detection.

The at least one chemical compound can “mimic” a feature of a cell receptor by providing for similar noncovalent interactions as would be provided by a naturally-occurring RBD/receptor interface. The noncovalent interactions can be similar in number, type, or both number and type. For example, the chemical compound can provide for a same or similar number of ionic interactions (e.g., salt bridges), hydrogen bonds, or both.

Cysteamine is a stable aminotiol (i.e., an organic compound that contains amine and thiol functional groups). Cysteamine can provide for mimicking the NH₂ functional group present on ACE2.

PBSE is an aromatic material that has been used for the detection of nucleic acids. PBSE is structurally similar to ACE2. In particular, PBSE includes a double bond oxygen functional group that is structurally similar to that present on ACE2.

The sensor can include, beneath or adjacent the electrocatalytic layer, a metal layer. The metal layer can comprise one or more layers of one or more metals. The one or more layers of metal can each be independently selected from a layer of chromium, platinum, gold, nickel, cobalt, tungsten, rhodium, iridium, silver, tin, titanium or tantalum, or an alloy thereof (e.g., nickel-tungsten, nickel-chromium).

The sensor can include a silicon substrate. Silicon substrates, such as silicon wafers, typically form a layer of silicon dioxide upon exposure to air, or come with a layer of silicon dioxide grown thereon, e.g., using one of a variety of oxidation processes known in the art (e.g., wet or dry oxidation). “A layer of silicon,” as used herein, is meant to encompass a layer of silicon including a layer of silicon dioxide. Typically, only one surface (e.g., top surface, bottom surface) of a layer of silicon includes a layer of silicon dioxide, but both surfaces (e.g., top surface and bottom surface) of a layer of silicon can include a layer of silicon dioxide. When a layer of silicon includes a layer of silicon dioxide, one or more layers of metal on the layer of silicon can be in physical contact with a silicon-containing surface. Alternatively, one or more layers of metal on a layer of silicon can be in physical contact with a silicon dioxide-containing surface.

The sensor can include two or more electrodes disposed at the MIP layer. Suitable materials for the electrodes include, but are not limited to, gold and silver.

A sensor comprising a MIP, an electrocatalytic layer, a metal layer, and, optionally, a silicon substrate can have an inherent resistance. For example, a resistance of the device in the absence of a template molecule or analyte can be from about 1 ohm to about 2,000 ohms, or from about 20 to about 1,000 ohms, or from about 100 to about 500 ohms.

In the presence of the template molecule or analyte, a resistance of the device can change. In particular, as an analyte molecule binds to the MIP layer, a resistance through polymer layer generally increases. A measured increase of about 10% to about 2000%, or of about 200% to about 1000%, or of about 20% to about 500% over the inherent resistance of the device, can be indicative of the presence of the analyte in a sample. The increase can depend upon a concentration of the analyte in the sample. As such, an amount of the analyte in the sample can be estimated based on a measured resistance.

A resistance of the device can be measured by an ohmmeter in operative arrangement with electrodes disposed at the MIP layer of the sensor, with a voltage source configured to apply a voltage to the polymer layer, as illustrated in FIG. 7.

The device can be exposed to a sample that is in the form of air or other fluid. The sample can be a biological sample (e.g., a breath, saliva, sweat, blood or urine sample, especially a breath sample, obtained from a subject). For example, the sample can be exhaled breath, which can contain droplets, aerosols, or both. The analyte can be present in the droplets and/or aerosols of the exhaled breath of a subject.

Functionalization of the electrocatalytic layer of a molecularly imprinted electrochemical sensor can significantly improve sensor performance. By mimicking the affinity between a virus and a cell receptor (e.g., between the SARS-CoV-2 virus and ACE2), sensitivity of the device can increase significantly (e.g., up to about 8000 times more sensitive than nonfunctionalized sensors), as further described in the following Examples.

The device can comprise one or more layers of metal (e.g., 1, 2, 3, 4, 5, etc.), optionally on a layer of silicon, and a layer of molecularly imprinted polymer in electrical communication with the one or more layers of metal. The one or more layers of metal are each independently selected from a layer of chromium, platinum, gold, nickel, cobalt, tungsten, rhodium, iridium, silver, tin, titanium or tantalum, or an alloy thereof (e.g., nickel-tungsten, nickel-chromium).

In some aspects, the layer of silicon dioxide is from about 1 nanometer to about 1 micron, from about 1 nanometer to about 500 nanometers, from about 1 nanometer to about 100 nanometers, from about 1 nanometer to about 50 nanometers or from about 1 nanometer to about 10 nanometers thick. In a particular aspect, the layer of silicon dioxide is about 5 nanometers thick.

Passivation refers to a material becoming passivate, that is, less affected or corroded by the environment. In an aspect, the metal, or alloy thereof, does not passivate upon exposure to air, or passivates upon exposure to air but does not develop a thick layer of oxide as a result of passivating. Metals that do not passivate upon exposure to air, or passivate upon exposure to air but do not develop a thick layer of oxide as a result of passivating include, but are not limited to, chromium, platinum, gold, nickel, cobalt, tungsten, rhodium, iridium, silver and tin. Accordingly, in some aspects, the one or more layers of metal are each independently selected from a layer of chromium, platinum, gold, nickel, cobalt, tungsten, rhodium, iridium, silver or tin, or an alloy thereof. In some aspects, the one or more layers of metal are each independently selected from chromium, nickel, cobalt, tungsten, rhodium, iridium, silver or tin, or an alloy thereof. In some aspects, the one or more layers of metal are each independently selected from chromium, or an alloy thereof.

In some aspects, the device comprises one layer of metal, and the metal is chromium, platinum, gold, nickel, cobalt, tungsten, rhodium, iridium, silver, tin, titanium or tantalum, or an alloy thereof. In a more particular aspect, the device comprises one layer of metal, and the metal is chromium, platinum, gold, nickel, cobalt, tungsten, rhodium, iridium, silver or tin, or an alloy thereof. In a yet more particular aspect, the device comprises one layer of metal, and the metal is chromium, nickel, cobalt, tungsten, rhodium, iridium, silver or tin, or an alloy thereof. In another aspect, the device comprises one layer of metal, and the metal is chromium, or an alloy thereof. In another aspect, the device comprises one layer of metal, and the metal is chromium.

In another aspect, the one or more layers of metal (e.g., one layer of chromium) is from about 5 microns to about 50 microns, from about 10 microns to about 25 microns or from about 10 microns to about 20 microns thick. In a particular aspect, the one or more layers of metal (e.g., one layer of chromium) is about 15 microns thick.

In yet another aspect, the layer of silicon is from about 50 microns to about 5 millimeters, from about 250 microns to about 1 millimeter, from about 250 microns to about 650 microns or from about 350 microns to about 400 microns thick. In some aspects, the layer of silicon is about 275 microns, about 375 microns, about 525 microns, about 625 microns, about 675 microns, about 725 microns, about 775 microns or about 925 microns thick. In a particular aspect, the layer of silicon is about 375 microns thick.

In another aspect, the one or more layers of metal (e.g., one layer of chromium) and the layer of silicon have a combined resistance of from about 1 ohm to about 1,000 ohms, from about 1 ohm to about 100 ohms, from about 10 ohms to about 100 ohms, from about 25 ohms to about 100 ohms, or from about 50 ohms to about 75 ohms. In a particular aspect, the one or more layers of metal (e.g., one layer of chromium) and the layer of silicon have a combined resistance of about 60 ohms.

In another aspect, the layer of molecularly imprinted polymer is from about 1 nanometer to about 5 millimeters, from about 1 nanometer to about 1 millimeter, from about 5 nanometers to about 500 nanometers, from about 10 nanometers to about 100 nanometers or from about 10 nanometers to about 50 nanometers thick. In a particular aspect, the layer of molecularly imprinted polymer is about 20 nanometers thick.

In another aspect, the resistance of the device is from about 1 ohm to about 1,000 ohms, from about 1 ohm to about 100 ohms, from about 10 to about 100 ohms, from about 25 ohms to about 100 ohms, or from about 50 ohms to about 75 ohms. In a particular aspect, the resistance of the device is about 60 ohms.

In another aspect, the device further comprises one or more (e.g., 1, 2, 3, 4, 5, 6, 7, 8, 10, 15, about 15, 20, about 20, 25, about 25, 50, about 50, 75, about 75, 100, about 100, 150, about 150, 200, about 200, 250, about 250, 300, about 300, 350, about 350, 400, about 400, 450, about 450, 500 or about 500) layers of graphene (e.g., reduced graphene oxide). In some aspects, the one or more layers of graphene (e.g., reduced graphene oxide) are between the layer of metal and the layer of molecularly imprinted polymer, the one or more layers of graphene being in electrical communication with the one or more layers of metal and the layer of molecularly imprinted polymer. Accordingly, an example device can comprise one or more layers of metal (e.g., one layer of metal) on a layer of silicon; one or more layers of graphene (e.g., one or more layers of reduced graphene oxide) on the one or more layers of metal; and a layer of molecularly imprinted polymer on the one or more layers of graphene. The one or more layers of graphene are in electrical communication with the one or more layers of metal and the layer of molecularly imprinted polymer.

When a device comprises one or more layers of graphene (e.g., reduced graphene oxide), the device comprises from about one to about 500, from about one to about 50, from about one to about 25, or from about 5 to about 15 layers of graphene. For example, the device comprises 1, 2, 3, 4, 5, 6, 7, 8, 10, 15, about 15, 20, about 20, 25, about 25, 50, about 50, 75, about 75, 100, about 100, 150, about 150, 200, about 200, 250, about 250, 300, about 300, 350, about 350, 400, about 400, 450, about 450, 500 or about 500 layers of graphene. In a particular aspect, the device comprises from about 5 to about 10 layers of graphene (e.g., reduced graphene oxide).

In some aspects, the analyte can be a virus or a portion of a virus, such as a protein present on a virus surface. In a particular aspect, the viral protein is a viral fusion protein, such as a glycoprotein. Example viruses include, but are not limited to, Ebola, severe acute respiratory syndrome coronavirus (SARS-CoV), influenza virus (e.g., H1N1 influenza virus), Middle East respiratory syndrome coronavirus (MERS-CoV), severe acute respiratory syndrome coronavirus 2 (SARS-CoV-2), human immunodeficiency virus (HIV), Dengue virus, Chikungunya virus, Hepatitus A virus, Hepatitus B virus, and Hepatitus C virus.

A method of detecting one or more analytes from a subject suspected to be infected with a virus is also provided. The method comprises measuring the resistance of a device described herein that has been or is in contact with a sample (e.g., a breath, sweat, saliva, blood or urine sample, especially a breath sample) obtained from the subject. The device has an inherent resistance, and is selective for one or more analytes associated with the virus. A difference between the inherent resistance of the device and the resistance of the device contacted with the sample indicates one or more analytes associated with the virus have been detected.

As used herein, “inherent resistance” refers to the resistance of a device described herein in the absence or substantial absence of the one or more analytes for which the device is selective. A person of ordinary skill in the art will appreciate that the methods described herein can be carried out by comparing the inherent resistance of the device (e.g., as an absolute number) to the resistance of the device contacted with a sample (e.g., also as an absolute number), such that the magnitude of the difference, if calculated, is the mathematical difference between the inherent resistance and the resistance of the device contacted with the sample. Alternatively, resistance can be based on a normalized scale, for example, using the following equation: [(R−Rₒ)/Rₒ]×100, where R is the resistance of the device contacted with a sample and Rₒ is the inherent resistance of the device. When normalized resistance is used, the device still has an inherent resistance associated with an absolute value, but the inherent resistance is assigned a value of zero. Using this method of comparing inherent resistance and resistance of a device contacted with a sample, the change in the resistance of the device (e.g., upon exposure to a sample) is the mathematical difference between the inherent resistance of the device and the resistance of the device contacted with the sample. The methods described herein contemplate and include both methods, and either understanding of “inherent resistance.”

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some aspects, the subject is a mammal. In some aspects, the subject is a human.

Methods of making the devices disclosed herein are also provided. A method of making a device comprises applying one or more layers of metal to a layer of silicon, applying one or more layers of an electrocatalyst, and applying a layer of molecularly imprinted polymer to the device such that the layer of molecularly imprinted polymer is in electrical communication with the one or more layers of metal. The method can further include functionalizing the electocatalytic layer(s) and, optionally, applying a dopant to the electrocatalytic layer. The one or more layers of metal are each independently selected from a layer of chromium, platinum, gold, nickel, cobalt, tungsten, rhodium, iridium, silver, tin, titanium or tantalum, or an alloy thereof. Molecularly imprinted polymers, electrocatalysts, functionalization materials, alternative metals, and other variations on the device are as described herein. In some aspects, the one or more layers of metal are sputtered onto the layer of silicon.

In some aspects, the method of making further comprises applying electrical contacts to the device such that the electrical contacts are in electrical communication with the layer of molecularly imprinted polymer. In some aspects, the electrical contacts are applied to the layer of molecularly imprinted polymer. Electrical contacts are as described herein.

### EXEMPLIFICATION

### Example 1. Functionalized Molecularly-Imprinted Electrochemical Sensors

In this study, the fabrication and testing of four sets of sensors to detect SARS1, SARS2, SARS-CoV-2, and Ebola virus is reported, along with the fabrication and testing of functionalized sensors for the detection of SARS-CoV-2. The spike proteins of SARS-CoV-2 (2 mutation), Ebola, and SARS were provided from the University of Massachusetts. Since the spike protein of the SARS-CoV-2 virus in isolation is identical to its form as included on the virus, the spike proteins were chosen instead of the whole virus.

The aim of this work was to evaluate sensitivity of the sensors.

### Sensor Fabrication Methods

An overview of the fabrication process is shown in FIG. 2. In brief, for each sensor, a highly doped silicon wafer was etched with hydrofluoric acid (HF) and coated with 15 nm of Chromium through sputtering. The wafer was then diced into squares of 5*5 mm². A solution of reduced graphene oxide and Prussian-blue was then injected onto the surface of the samples. The molecular-imprinting electrolyte was prepared through dissolving one specific spike protein in a mixture of phosphate buffer solution and pyrrole. These components of the fabrication process of the gas sensors is as described in more detail in U.S. Pub. No. 2019/0313944, the entire teachings of which are incorporated herein by reference.

The functionalization process was performed after the deposition of graphene-Prussian blue. First, the sensor was soaked in 2 mM PBSE (Sigma Aldrich) in methanol for 1 h at room temperature and then rinsed several times with deionized water. Then, a 25*10−3 mol/L solution of cysteamine was incubated at the surface of the sensor for 2 minutes at room temperature.

The functionalization of the graphene layer to include both cysteamine and PBSE is referred to as “double functionalization.”

### Concentration Estimates

The virus copies that are spread by a subject with/without wearing a mask was experimentally estimated. See Leung, N. H. L., Chu, D. K. W., Shiu, E. Y. C. et al. Respiratory virus shedding in exhaled breath and efficacy of face masks. Nat Med 26, 676-680 (2020). Assuming the virus can spread both through droplet and aerosol, an average of 123010 copies of virus is estimated to be present in exhaled breath. Considering the volume of each exhaled breath as 1 L, a concentration of the virus in exhaled breath is estimated to equal 0.5 parts per quadrillion. The concentrations of the testing samples were 10 and 100 ng/μl. However, concentration of the protein in the air was also calculated. Considering that proteins are not volatile, combing Maxwell-Boltzmann distribution of velocities and amount leaving the surface, the vapor pressure of a non-volatile substance can be derived. See Clyde, D. Dean., Svec, H. J., Vapor pressures of some amino acids. Ames Laboratory., U.S. Atomic Energy Commission. (1963). With this method, the vapor pressure of the spike protein was calculated to be 12 parts per quadrillion.

### Results—Non-Functionalized Sensors

Four sensors were fabricated, without functionalization (i.e., without cysteamine and/or PBSE), and tested to detect SARS-CoV-2 D614D and D614G mutations, SARS1, and Ebola. Each sensor was given 6 minutes in the absence of any stimulant to monitor its stability. In the next 2 minutes, the sensors were exposed to the vapor of 10 ng (in 1 μl) of their own spike protein. For the latter 2 minutes, the sensors were exposed to the vapor of 100 ng (in 1 μl) of the spike protein, and then the sensors were given 2 extra minutes to return to their base resistance. The normalized resistance was derived based on the following:

$${R}_{Normalized} = [ \frac{R - {R}_{0}}{{R}_{0}} ] \times 100. \qquad ( 1 )$$

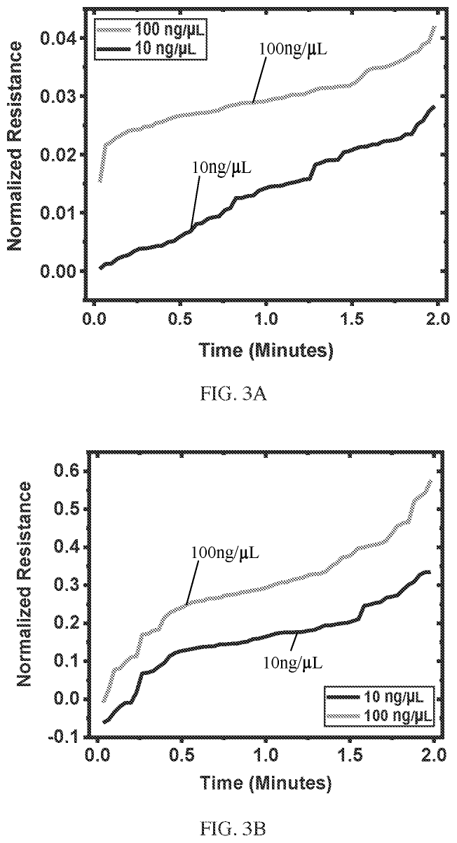

The results of each sensor are shown in FIGS. 3A-3D. The graphs of FIGS. 3A-3D illustrate measurements obtained during the two-minute timeframes at which the sensors were exposed to the vapor.

### Results—Double-Functionalized Sensors

Two sensors for detecting SARS2-D614D and SARS2-D614G were fabricated with the functionalization method. After the deposition of graphene, the sensors were dipped in a solution of PBSE and methanol for 1 hour and then buried in a solution of cysteamine and methanol for a few seconds, as described in the methods above. As compared to the non-functionalized sensors, noticeable changes in terms of resistance were observed. While the resistance change of the non-functionalized sensors was in the order of milliohms, one functionalized sensor showed a resistance change of about 400 Ohms upon exposure to 10 ng/μL of spike-protein solution, which demonstrates an improvement over first generation, nonfunctionalized sensors of about 2000×.

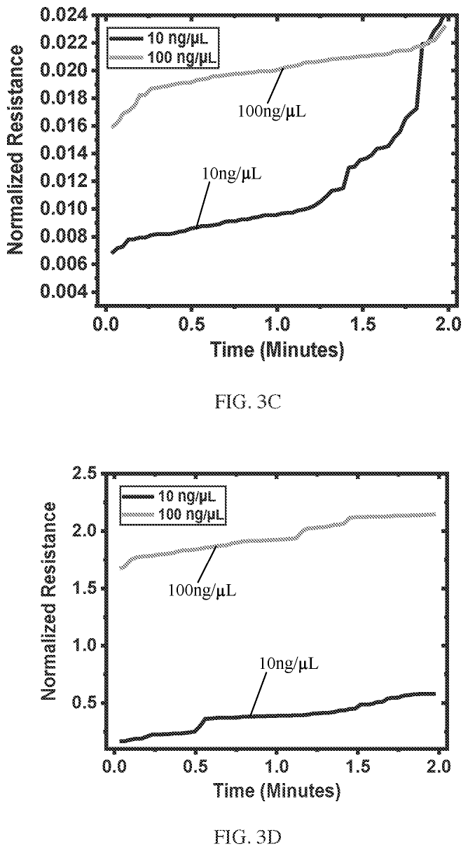

The results of each sensor are shown in FIGS. 4A-4B. Both sensors demonstrated recovery. FIG. 4B illustrates the sensor recovery upon an end of exposure to the spike-protein solution.

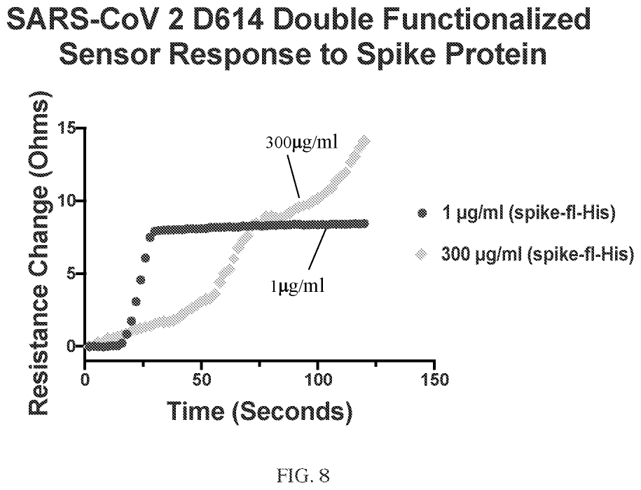

Additional results of another double-functionalized sensor for detection of SARS2-D614D are shown in FIG. 8. The sensor was exposed to spike protein concentrations of 1 μg/ml and 300 μg/ml, and changes in resistance of about 8 to about 15 ohms were observed.

### Results—Cysteamine-Functionalized Sensor at Two Concentrations

A sensor for detecting SARS2 was fabricated with a functionalization method involving only cysteamine (no PBSE). After the deposition of graphene, the sensors were buried in a solution of cysteamine and methanol for a few seconds, as described in the methods above but without the inclusion of PBSE.

The sensor was then exposed to two concentrations of the spike protein. As compared to the non-functionalized sensors, the double-functionalized sensors had noticeable changes in terms of resistance change. The cysteamine-functionalized sensor showed a greater change in resistance than the non-functionalized sensors, but less than that of the double-functionalized sensors.

### Results—Comparison of Non -Functionalized Sensor and Cysteamine-Functionalized Sensor with Heat-Inactivated Virus

Heat-inactivated SARS-CoV-2 ATCC® VR-1986HK™ was purchased from ACTT. This product is a preparation of Severe acute respiratory syndrome-related coronavirus 2 (SARS-CoV-2) strain 2019-nCoV/USA-WA1/2020 that has been inactivated by heating to 65° C. for 30 minutes and is therefore unable to replicate. The vial contained approximately 0.25 mL of heat-inactivated, clarified cell lysate and supernatant from Vero E6 cells infected with SARS-CoV-2 strain 2019-nCoV/USA-WA1/2020. So, the concentration of this virus cannot be determined.

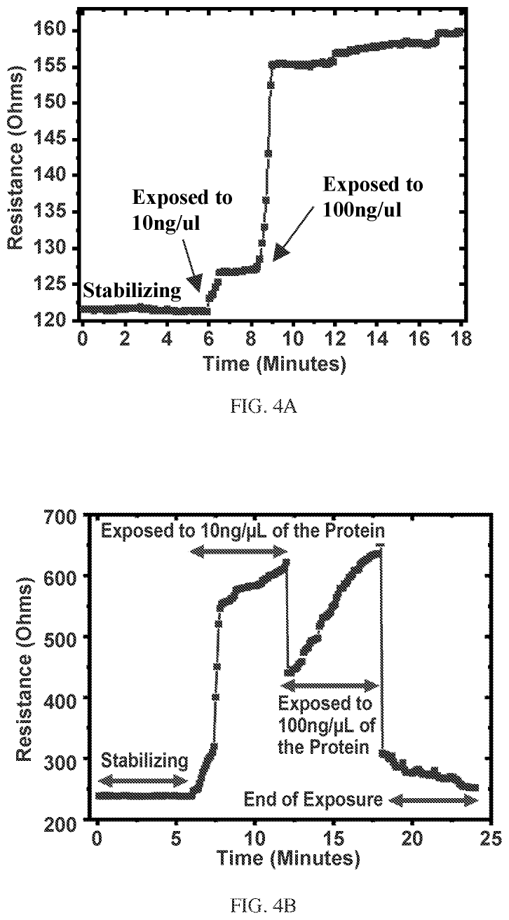

Two sensors were fabricated and tested with this virus and the results are shown in FIGS. 5A-5B. In particular, FIG. 5A illustrates the results of the sensor fabricated without functionalization, and FIG. 5B illustrates the results of the sensor fabricated with the functionalization method with cysteamine (without PBSE). The normalized resistance of the sensors was monitored while exposed to the sensors for 6 minutes in a 1 L sealed chamber. The cysteamine-functionalized sensor was an order of magnitude more sensitive to the virus than the non-functionalized sensor.

### Results—Sensitivity

The SARS-CoV-2 gas sensors were fabricated with cysteamine and PBSE for sensor functionalization and tested to detect SARS-CoV-2 D614D and D614G mutation strains (FIGS. 4A-4B). During the sensor tests, the sensors were first given 6 minutes in the absence of any stimulant to monitor their stability. In the next 6 minutes, the sensors were exposed to the vapor of 10 ng (in 1 μL) of their own spike protein. For the next 6 minutes, the sensors were exposed to the vapor of 100 ng (in 1 μL) of the spike protein, and then the sensors were given 6 extra minutes to return to their base resistance. As shown in FIG. 4B, for the D614G mutation, the sensor demonstrated a large resistance change (dR) of 400 Ohm in a short time with signs of sensor saturation at an elongated time of sensor exposure to 10 ng/μL full-length spike proteins.

For sensitivity tests, the resistance change (dR) of the SARS-CoV-2 sensor when the sensor was exposed to the vapor of the full-length spike protein solution with a concentration (C) for different viruses was monitored. The limit of detection of the Ohm-meter for the sensor system (LoD_R) is at LoD_R=0.1 mOhm. The sensitivity of these SARS-CoV-2 sensors is defined as C/dR. The LoD for the gas sensors for sensing spike proteins in the air for corresponding spike protein concentrations in liquid (LoD_C) for the SARS-CoV-2 sensors can be estimated as LoD_C=LoD_R*C/dR, which is the LoD for spike protein concentration in liquid, such as human saliva during real COVID-19 tests.

The SARS-CoV-2 sensor showed a resistance change dR=400 Ohms when exposed to a 10 ng/μL solution of full-length spike proteins of SARS-CoV-2, corresponding to a concentration of ˜20 nM as a trimer (or ˜60nM as a monomer). The signal to noise ratio of such a measurement is dR/LoD_R=4,000,000, or 132 dB. Therefore, the limit of detection for the full-length spike proteins of SARS-CoV-2 is LoD_C=LoD_R*C/dR=0.1 mOhm*20 nM/400 Ohms=5×10⁻¹⁵M in liquid.

Human saliva for COVID-19 patients has a viral load of 10³˜10¹¹ copies/mL, according to Wyllie et al. (Wyllie et al. Saliva is more sensitive for SARS-CoV-2 detection in COVID-19 patients than nasopharyngeal swabs. medRxiv 2020), while the gold standard RT-PCR for COVID-19 diagnosis has an LoD of 10³˜10⁴ copies/mL. One SARS-CoV-2 virus has ˜1000 spike proteins, which leads to spike protein concentrations of 10⁹˜10^{17/}L for the saliva of COVID-19 patients, i.e., 10⁻¹⁵˜10⁻⁷ M for spike protein concentrations in saliva. Therefore, the fabricated SARS-CoV-2 sensor with a spike protein concentration limit of detection of 5×10⁻¹⁵ M in liquid can measure the COVID-19 patients with their saliva having a spike protein concentration of 5×10⁻¹⁵ M˜10⁻⁷ M, or a corresponding SARS-CoV-2 virus load range of 3×10³˜10¹¹ copies/mL. The LoD of 3×103 copies/mL for these gas sensors is comparable to that of RT-PCT, or corresponding to being able to detect ˜100% of all the COVID-19 patients by our gas sensor, or ˜100% sensitivity.

The gas sensors can be applied to diagnose COVID-19 from the exhaled breath instantly. An array of four sensors were fabricated with the spike proteins of four viruses including: SARS2, SARS2-D614G, SARS1, and Ebola. Each sensor was tested against both low and high concentration of the same virus. To increase the sensitivity of the sensors, the sensors were functionalized to mimic the affinity between the SARS-CoV-2 virus and ACE2. With the functionalization method, the sensitivity of the sensors increased up to 8000 times. Finally, the sensors were tested against the heat-inactivated virus. Each sensor was only sensing the one specific virus for which it was made, and the sensitivity of the sensors was measured by the resistance change of the sensors, which can be easily monitored with, e.g., a multimeter. Sensors, as described herein, can be a substitute for the current diagnosing method for the SARS-CoV-2 virus (and the other viruses), which is time consuming, costly, and can provide relatively high false positive results. If implemented, these sensors can be applied to diagnose COVID-19 instantly, non-invasively, fast, and accurately.

While example embodiments have been particularly shown and described, it will be understood by those skilled in the art that various changes in form and details may be made therein without departing from the scope of the embodiments encompassed by the appended claims.

The teachings of all patents, published applications and references cited herein are incorporated by reference in their entirety.

1. A sensor, comprising:
a polymer layer molecularly imprinted for an analyte;
a metal layer;
an electrocatalytic layer disposed between the polymer layer and the metal layer, the electrocatalytic layer functionalized with at least one chemical compound that provides for noncovalent interaction with the analyte; and
electrodes in operative arrangement with the polymer layer and configured to provide a signal indicative of a resistance.

2. The sensor of claim 1, wherein the analyte is a receptor-binding domain of a virus.

3. The sensor of claim 2, wherein the analyte is a glycoprotein.

4. The sensor of claim 3, wherein the glycoprotein is a glycosylated spike protein.

5. The sensor of claim 4, wherein the glycosylated spike protein binds to an angiotensin-converting enzyme 2 (ACE2) receptor.

6. The sensor of claim 2, wherein the virus is SARS1, SARS2, SARS-CoV-2, or Ebola.

7. The sensor of claim 6, wherein the SARS-CoV-2 virus is SARS-D614D or SARS-D614G.

8. The sensor of claim 1, wherein the electrocatalytic layer comprises graphene.

9. The sensor of claim 8, wherein the electrocatalytic layer further comprises potassium ferrocyanide.

10. The sensor of claim 1, wherein the analyte is a protein and the at least one chemical compound mimics a feature of a cell receptor for the protein.

11. The sensor of claim 10, wherein the cell receptor is an angiotensin-converting enzyme 2 (ACE2) receptor.

12. The sensor of claim 1, wherein the at least one chemical compound comprises 1-pyrenebutyric acid n-hydroxysuccinimide ester (PBSE).

13. The sensor of claim 1, wherein the at least one chemical compound comprises cysteamine.

14. The sensor of claim 1, wherein the metal layer comprises chromium.

15. The sensor of claim 1, further comprising a silicon substrate.

16. A detector comprising:
the sensor of claim 1; and
a voltage source configured to apply a voltage to the polymer layer.

17. The detector of claim 16, further comprising an ohmmeter in operative arrangement with the electrodes and configured to output a measurement of the resistance.

18. A method of detecting an analyte, comprising:
measuring a resistance of a molecularly imprinted polymer exposed to a sample, the molecularly imprinted polymer imprinted for the analyte and in operative arrangement with an electrocatalyst and a metal, the electrocatalyst functionalized with at least one chemical compound that provides for noncovalent interaction with the analyte, the electrocatalyst disposed between the molecularly imprinted polymer and the metal, the measured resistance indicative of a presence of the analyte in the sample.

19. The method of claim 18, wherein the sample is in an aerosol or droplet form in exhaled breath.

20. The method of claim 18, further comprising comparing the measured resistance to an inherent resistance of the molecularly imprinted polymer in arrangement with the electrocatalyst and the metal.

21. The method of claim 20, wherein a greater measured resistance than the inherent resistance indicates the analyte is present in the sample.

22. The method of claim 20, wherein a difference between the measured resistance and the inherent resistance indicates an amount of the analyte present in the sample.

23. A method of detecting an analyte, comprising:
exposing the sensor of claim 1 to a sample; and
measuring the resistance of the device as the polymer layer is exposed to the sample to obtain a measured resistance, wherein the measured resistance is indicative of a presence of the analyte in the sample.

24. The method of claim 23, wherein the sample comprises an aerosol or droplets from exhaled breath.

25. A method of detecting a viral infection in a subject, comprising:
exposing the sensor of claim 1 to a sample obtained from the subject, wherein the analyte is a receptor-binding domain of a virus; and
measuring the resistance of the device as the polymer layer is exposed to the biological sample to obtain a measured resistance, wherein the measured resistance is indicative of the viral infection in the subject.

26. A method of manufacturing a sensor, comprising:
depositing an electrocatalytic layer onto a metal layer;
functionalizing the electrocatalytic layer with at least one chemical compound that provides for noncovalent interaction with an analyte of interest; and
forming a molecularly imprinted polymer on the electrocatalytic layer, the molecularly imprinted polymer imprinted for the analyte.
